(19) United States
(12) Patent Application Publication
(10) Pub. No.: US 2014/0274891 A1
(43) Pub. Date: Sep. 18, 2014

(54) METHOD OF GENERATING AN INDEX SCORE FOR MBL DEFICIENCY TO PREDICT CARDIODIABETES RISK

(71) Applicant: Health Diagnostic Laboratory, Inc., Richmond, VA (US)

(72) Inventors: Rebecca E. Caffrey, North Chesterfield, VA (US); James Pottala, Sioux Falls, SD (US); Stephen Varvel, Richmond, VA (US)

(73) Assignee: Health Diagnostic Laboratory, Inc., Richmond, VA (US)

(21) Appl. No.: 14/216,850

(22) Filed: Mar. 17, 2014

Related U.S. Application Data
(60) Provisional application No. 61/794,450, filed on Mar. 15, 2013.

(51) Int. Cl. G01N 33/566; A61K 38/17

(52) U.S. Cl. CPC G01N 33/566; A61K 38/1709
USPC 514/6.9; 514/16.4; 435/7.92; 435/7.4; 702/19

(57) ABSTRACT

This application relates to methods of predicting susceptibility or likelihood of a clinically-relevant mannose-binding lectin (MBL)-deficient subject to develop a cardiovascular disease and/or cardiodiabetes. The methods include measuring MBL mass or concentration and, optionally, measuring MBL activity, at least one other biomarker and/or genotyping of MBL gene and its promoters; combining the information obtained into a calculated MBL-inclusive index score that involves mathematical transformation; and assigning a risk of cardiadiabetic status and clinical endpoints based on the determination and comparison of the MBL inclusive index to reference values from a population.

### CROSS-REFERENCE TO RELATED APPLICATIONS

This application claims the benefit of priority to U.S. Provisional Patent Application Ser. No. 61/794,450, filed Mar. 15, 2013, which is hereby incorporated by reference in its entirety.

### FIELD OF THE INVENTION

This application relates to methods of predicting susceptibility or likelihood of a clinically-relevant mannose-binding lectin (MBL)-deficient subject to develop a cardiovascular disease and/or cardiodiabetes.

### BACKGROUND

Mannose Binding Lectin (MBL) is the plasma protein that binds to proteins that have been glycated with mannose (or mannan), and especially those on the bacterial cell walls. MBL activates the complement cascade via the lectin pathway and is important in the innate immune response. MBL helps or “complements” the ability of antibodies and phagocytic cells to clear pathogens from an organism. The MBL pathway of complement activation is the third pathway for activation of this cascade. As a serum protein, MBL binds carbohydrate residues and circulates in the serum in complex with a type of serine protease protein called mannan-binding lectin associated serine proteases (MASPs). When the MBL complex binds to carbohydrate residues (mannose residues on bacterial cell walls, for instance), the MBL complex activates complement components, C4 and C2, thus generating the C3 convertase and leading to deposition of the generated fragments, C4b and C3b (see Hamad, I. et al., 2008, which is hereby incorporated by reference in its entirety). This activation process promotes opsonization of the micro-organisms and can assist with the clearing of infections.

Normal human plasma contains an MBL concentration ranging from 33 to 1650 U/ml. About 12% of (apparently) healthy Caucasian blood donors have MBL levels below 33 U/ml. Because MASP protein occurs in vast excess to the amount of MBL, the MBL is bound up in MASP complexes. If all humans had the same MBL activity, then the deposition of C4b (C4b depositing capacity in an assay) would be the same for people with the same measured amount of MBL. However, this may not be the case. The C4b deposition capacity varies significantly (3-fold) between individuals with similar MBL concentration (see Petersen, S. et al., 2001, which is hereby incorporated by reference in its entirety). Therefore, one can be “immunodeficient” due to insufficient amounts of MBL, insufficient activity, or both. Similarly, one can be at risk of diseases from excessive amounts of MBL particularly when that excess MBL functions optimally, whereas an excess amount of MBL that does not function optimally may not be detrimental.

MBL deficiency is one of the most frequent immunodeficiencies that affect approximately 10% of the general population. MBL deficiency is associated with inflammation, infections, development of gestational diabetes mellitus (GDM), development of vasculitis, arterial stiffness in Kawasaki Disease (see Biezeveld, M. H. et al., 2003, which is hereby incorporated by reference in its entirety) and has been associated with the appearance of early insulin resistance, early atherosclerosis and more progressive forms of atherosclerosis (see Megia, et al., 2004, which is hereby incorporated by reference in its entirety). MBL deficiency has also been linked to increased risk of Epstein-Barr viral infection and increased chance of invasive pneumococcal infection, whereas excessive MBL increases risk of cardiovascular events leading to mortality in Rheumatoid Arthritis (RA), increased chance of arterial thrombosis in Systemic Lupus Erythematosus (SLE) for some genotypes, and recurrent late pregnancy losses. Both insufficient and excessive levels of MBL may result in dysregulation of the system because MBL plays such a central role in hemostasis, immunity, and inflammation.

While much of the literature regarding MBL and immunodeficiency and cardiodiabetes risk focuses on its role in complement cascade, little attention has been paid to the fact that MBL can bind lipoproteins. MBL has been shown to bind to LDL and enhance the monocyte/macrophage clearance of LDL. MBL is also known to enhance HDL-mediated cholesterol efflux from macrophages (see Fraser, D. A. and Tenner, A. J., 2010, which is hereby incorporated by reference in its entirety). This function may be part of the component of cardiovascular risk association. Clearance of LDL and the ability of macrophages to export cholesterol to HDL (cholesterol efflux) are critical processes for lipid homeostasis in the blood vessel walls; if one or both of these are compromised, cardiovascular disease (and particularly atherosclerosis) result. It could be inferred from the background information above that a sufficient amount of MBL with sufficient activity would promote proper function and balance in LDL clearance and HDL-mediated cholesterol efflux from macrophages. However, studies have not been done so far to clarify the synergism of MBL amount and activity on cardiovascular disease development from these processes in vivo.

There is a need for a method wherein patients are screened for absolute amounts of MBL (MBL mass) in the serum and biological activity level of their MBL protein, as well as MBL genotyping including the MBL promoter region to determine whether these patients have clinically-relevant MBL deficiency to get them the most appropriate therapy before coronary artery disease (CAD) develops. There is also a need for a method to combine the measurements of MBL mass and MBL activity, with an index derived therefrom with additional biomarkers to predict susceptibility or likelihood of the patients who are MBL-deficient to develop cardiovascular diseases or cardiodiabetes. This invention answers these needs.

### SUMMARY OF THE INVENTION

This invention relates to a method for predicting susceptibility or likelihood of a subject having a clinically-relevant mannose-binding lectin (MBL) deficiency to develop cardiodiabetes. The method includes the following steps: (a) obtaining a measurement value of MBL mass and, optionally, a measurement value of MBL activity level; (b) calculating an MBL-inclusive index score based one or both MBL measurements, wherein the index score calculation involves a mathematical transformation; and (c) comparing the MBL-inclusive index to reference values from a population, wherein an elevated MBL-inclusive index score correlates with a range in a higher unit of an ordered distribution of the population and indicates that the subject is less susceptible to or has a less likelihood of developing cardiovascular disease and/or cardiodiabetes, and wherein a low MBL-inclusive index score correlates with a range in a lower unit of an ordered distribution of the population and indicates that the subject is more susceptible to or has an increased likelihood of developing cardiovascular disease and/or cardiodiabetes.

This invention also relates to a method for predicting susceptibility or likelihood of a subject having a clinically-relevant mannose-binding lectin (MBL) deficiency to develop cardiodiabetes. The method includes the following steps: (a) obtaining a measurement value of MBL mass and, optionally, a measurement value of MBL activity level; (b) obtaining a measurement value for at least one other biomarker; (c) calculating an MBL-inclusive index score based one or both MBL measurements and the at least one other biomarker, wherein the index score calculation involves a mathematical transformation; and (d) comparing the MBL-inclusive index to reference values from a population, wherein an elevated MBL-inclusive index score correlates with a range in a higher unit of an ordered distribution of the population and indicates that the subject is less susceptible to or has a less likelihood of developing cardiovascular disease and/or cardiodiabetes, and wherein a low MBL-inclusive index score correlates with a range in a lower unit of an ordered distribution of the population and indicates that the subject is more susceptible to or has an increased likelihood of developing cardiovascular disease and/or cardiodiabetes.

The mathematical transformation of the MBL-inclusive index score involves multiplication, division, logarithmic transformation, raising to a power, or any combination thereof.

An elevated or low MBL-inclusive index score can be classified into tertiles and a score in an upper tertile or lower tertile may indicate that the subject is either less or more susceptible to or has a less or an increased likelihood of developing cardiovascular disease and/or cardiodiabetes, respectively.

The MBL mass can be measured by enzyme-linked immunosorbent assay (ELISA), electrophoresis, double-enzyme immunoassay, immunofluorometry, and/or hemolytic assay.

The MBL activity level can be measured by ELISA, complement assay and/or mannan capture method assay or by one or more techniques selected from the group consisting of hemolysis assay, mannan capture assay, micro-organism lysis assay, an assay measuring ability to promote opsonization of a particle or micro-organism, and an assay measuring the production of complement components C4b and/or C3b.

A low MBL-inclusive score indicates a clinically-relevant MBL deficiency that may be associated with the development of an inflammation, an infection, gestational diabetes, prevalent diabetes, an autoimmunity, a complication from an autoimmune condition or infection, a blood clotting abnormality, an impaired glucose tolerance, an impaired first-phase insulin secretion response, compromised pancreatic beta cell dysfunction, an early insulin resistance, or any form of atherosclerosis. In addition, a clinically-relevant MBL deficiency may also identify a subject at risk for cardiodiabetes, atherosclerosis, heart attack or stroke.

Examples of the at least one other biomarker maybe selected from the group consisting of 1,5 AG; Adiponectin; Alpha hydroxybutyrate; Amylase; Apo B; Apo B/ApoA1 ratio; ApoB-48; apolipoprotein B-48 (ApoB-48); BMI; CD26; C-peptide; C-peptide/Insulin Ratio; C-peptide/Proinsulin ratio; C-reactive protein; Ferritin; Fibrinogen; Free Fatty Acids; Fructosamine; Functional MBL/MASP-2 Ratio; glucagon-like peptide 1 (GLP-1); Glucose; Glycation Gap; HbA1c; HDL cholesterol; HDL2 levels; HDL-C; HOMA Insulin Resistance Score; Insulin; Insulin Resistance Score; LDL cholesterol; LDL particle number; LDL Triglycerides; LDL-C; Leptin; Leptin/Adiponectin Ratio; Leptin/BMI ratio; linoleoyl-glycerophosphocholine (L-GPC); LpPLA(2); Mannose; MBL Mass; MBL/MASP2 Function Ratio; Myeloperoxidase (MPO); OGTT Index; Oleic Acid; Proinsulin; Proinsulin/C-peptide Ratio; Remnant-like lipoprotein particles (RLPs); RLP-associated cholesterol (RLP-c); small, dense LDL levels (sdLDL); Total Cholesterol; and Triglycerides.

In one embodiment, the MBL-inclusive index score includes the measurements for both MBL mass and MBL activity level. It may further includes the measurements for fructosamine, C-peptide, and 1, 5 AG.

In another embodiment, the MBL-inclusive index score includes the calculation:

$$LN [ \frac{MBL mass * 1 , 5 {AG}^{1.91}}{MBL activity * {Fructosamine}^{10.67} * C \text{-} {peptide}^{2.29}} ]$$

and can be calculated by (a) dividing the measurement value of MBL mass with the measurement value of MBL activity level; (b) mathematically incorporating the measurement of at least one other biomarker; and (c) logarithmically transforming the outcome generated from the dividing and mathematically incorporating steps.

The method also includes the step of screening for a genotype in an MBL coding sequence and its promoter region. It may also further include measuring the amount of an MBL-binding serine protease and/or genotyping MASP coding and/or promoter regions.

The susceptibility or likelihood of the subject to have cardiovascular disease and/or cardiodiabetes may be low, medium or high.

A high MBL-inclusive index score may also indicate a cardiovascular disease in a subject that has an autoimmune disease or condition.

The method may further include administering a therapeutic regimen for the treatment or prevention of cardiovascular disease or cardiodiabetes. A therapeutic regimen may be selected from the group consisting of (i) administration of a recombinant human MBL, plasma-derived MBL or an MBL analogue and/or inhibitor; (ii) administration of lipid-modulating compounds such as statins and PCSK9 inhibitors for aggressive management of LDL and Apo-B; (iii) diet and lifestyle intervention; (iv) administration of antibiotics and/or anti-viral agents; (v) administration of immuno-modulating therapies; (vi) administration of coagulation therapies; (vii) administration of therapeutics that modify the complement cascade; (viii) an antihypertensive therapy; (ix) an anti-diabetic therapy; (x) other drug-based and lifestyle-based therapeutic interventions; and a combination thereof.

The therapeutic regimen may further includes administration of drugs or supplements; treatment for chronic infections; referral to a healthcare specialist or related specialist based on the determination of the risk levels; recommendations on making or maintaining lifestyle choices; and a combination thereof.

The drugs or supplements may be selected from the group consisting of an anti-inflammatory agent, an anti-thrombotic agent, an anti-platelet agent, a fibrinolytic agent, a lipid-reducing agent, a direct thrombin inhibitor, a glycoprotein IIb/IIIa receptor inhibitor, a calcium channel blocker, a beta-adrenergic receptor blocking agent, an angiotensin-system inhibitor, angiotensin (renin-angiotensin) system inhibitor, a cellular adhesion molecule binding agent, an inhibitor of white blood cells to attach to a cellular adhesion molecule binding agent, a PSKC inhibitor, an MTP inhibitor, mipmercin, a glitazone, a GLP-1 analog, thiazolidinedionones, biguanides, neglitinides, alpha glucosidase inhibitors, an insulin, a dipeptidyl peptidase IV inhibitor, metformin, a sulfonurea, peptidyl diabetic drugs and combinations thereof.

The invention also relates to a method for predicting susceptibility or likelihood of a subject having a clinically-relevant mannose-binding lectin (MBL) deficiency to develop cardiodiabetes, comprising: (a) obtaining measurement values of MBL mass and MBL activity level; (b) obtaining measurement values for Fructosamine, C-peptide, and 1, 5 AG; (c) calculating an MBL-inclusive index score based the measurements obtained in steps (a) and (b) using the following equation:

$$LN [ \frac{MBL mass * 1 , 5 {AG}^{1.91}}{MBL activity * {Fructosamine}^{10.67} * C \text{-} {peptide}^{2.29}} ] ;$$

and (d) comparing the MBL-inclusive index to reference values from a population, wherein an elevated MBL-inclusive index score correlates with a range in a higher unit of an ordered distribution of the population and indicates that the subject is less susceptible to or has a less likelihood of developing cardiovascular disease and/or cardiodiabetes, an wherein a low MBL-inclusive index score correlates with a range in a lower unit of an ordered distribution of the population and indicates that the subject is more susceptible to or has an increased likelihood of developing cardiovascular disease and/or cardiodiabetes.

Additional aspects, advantages and features of the invention are set forth in this specification, and in part will become apparent to those skilled in the art on examination of the following, or may learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIG. 1 is a heat map display showing the absolute value of the correlation between the values of each biomarker and each cluster component score.

FIG. 2 is a histogram showing the measurement values of MBL mass (concentration).

FIG. 3 is a histogram showing the log(MBL Mass).

FIG. 4 is a histogram showing the measurement values of MBL activity (MBL/MASP2 complex).

FIG. 5 is a histogram showing the log(MBL Activity).

FIG. 6 shows a plot of Pearson correlations between 1-hour and 2-hour glucose measures with MBL mass and MBL mass/activity ratio.

FIG. 7 shows an ROC curve from a multivariable logistic regression model.

FIG. 8 shows a probability plot from a multivariable logistic regression model.

### DETAILED DESCRIPTION OF THE INVENTION

MBL deficiency has been correlated with the severity of atherosclerotic disease (see Madsen, H. O. et. al., 1998, which is hereby incorporated by reference in its entirety), and human population studies showed that higher levels of MBL were associated with decreased risk of MI (myocardial infarction) in hypercholesterolemic individuals (see Saevarsdottir, S. et al., 2005, which is hereby incorporated by reference in its entirety). The HUNT2 study in a Norwegian population found that MBL deficiency doubled risk of MI (see Vengen, I. T. et al., 2012, which is hereby incorporated by reference in its entirety). MBL/MASP-1/3 complexes have been shown together to mediate coagulation-factor like activities, similar to thrombin. Knock-out studies in mice have shown that MBL-null and/or MASP-1/3 null mice develop disseminated intravascular coagulation (DIC), oftentimes with liver injury, when infected with Staphylococcus aureus (see Takahashi, K., 2011, which is hereby incorporated by reference in its entirety). Therefore, MBL deficiency may predispose humans to enhanced clotting, contributing to morbidity and mortality from cardiovascular disease seen in studies.

Specific genotypes of MBL are known to confer susceptibility to or resistance to atherosclerosis as well as infections, such as C. pneumonia, a gram negative organism which is known to also initiate and accelerate the progression of atherosclerosis. In fact, humans with MBL deficiencies tend to have recurring C. pneumonia infections, and other infections, due in part to MBL's role in normal innate immunity (complement cascade initiation). One study found that patients with severe atherosclerosis had a reduced frequency of the MBL-A allele and an increased frequency of the MBL-B, -C, and -D alleles compared with apparently healthy controls (see Madsen, H. O. et. al., 1998, which is hereby incorporated by reference in its entirety). Other studies have found that populations like Inuit Canadians who have remarkably low levels of atherosclerosis and also higher resistance to C. pneumonia infections have much higher allele frequency of the functional wild-type MBL-A alleles (see Hegele, R. et al., 1999, which is hereby incorporated by reference in its entirety). Polymorphisms in the MBL gene promoter (termed H, L, X, and Y) also contribute to the MBL deficiency syndrome (see Madsen, H. O. et al., 1995 and Salimans, M. M. M. et. al, 2004, both of which are hereby incorporated by reference in their entirety). It is the interplay of these alleles in the MBL gene itself and the promoter region that determines the amount of the protein expressed in the blood and the functionality (activity) of the MBL.

Only seven haplotypes (out of a possible 64) are commonly found combining to form 28 genotypes (see Garred, P. et al., 2009, which is hereby incorporated by reference in its entirety). In disease association studies, these genotypes are usually grouped into assumed low (YO/YO and YO/XA), medium (YA/YO and XA/XA) and high (YA/YA and YA/XA) conferring categories (see Wallis, R. and Lynch, N. J., 2007, which is hereby incorporated by reference in its entirety). Most, but not all, individuals with A/A genotypes have serum MBL>600 ng/mL and those with O/O genotypes generally have serum MBL below 200 ng/mL (see Swierzko, A. S. et al., 2009, which is hereby incorporated by reference in its entirety). The A/O groups, however, are highly heterogeneous with respect to serum MBL values, despite average values being reported at ˜400 ng/mL and perhaps a majority having concentrations<600 ng/mL. (see Chalmers, J. D. et al., 2011, which is hereby incorporated by reference in its entirety).

MBL deficiency can be thought of as a combination of not enough MBL mass (concentration), and/or insufficient MBL activity (function), combined with other characteristics of a given patient's individual genetic make-up, comorbidities, diet and lifestyle that influence that individual's physiology and metabolism. Excess or overabundance of MBL can be thought of as arising from the interplay of the same factors enumerated above, but rather with high mass and/or high activity. Despite the fact that MBL deficiency is so common in most human populations (10% on average), it is rarely diagnosed because it is not a condition that is often screened for, except in the case of extremely sick infants with recurrent infections. Therefore, the vast majority of people who are at-risk for early-onset or especially aggressive cardiovascular disease, and other conditions associated with MBL deficiency, may have no idea that they are at-risk. One reason that the recombinant MBL therapy is not used often is that people are not screened; even if they were to be screened genetically, some studies show that heterozygotes with defective genes are symptomatic, and others show that homozygotes only are symptomatic and affected. Further confounding the picture is that people with genotypes who “could” be MBL-deficient have normal levels of the protein in their plasma and do not have symptoms of the disease, underscoring the point that other risk factors clearly may play a significant role in the pathology of conditions associated with MBL deficiency.

The discordance between studies and difficulty in predicting who has a functional MBL deficiency and can be therefore at-risk for a host of health issues but most particularly cardiodiabetes and atherosclerosis, heart attacks, and strokes, arises because the studies measure different things related to MBL and thus their results differ from one to another. Some studies only measure genetic variation, or amount of MBL in the plasma, or activity of the MBL. Further confounding the literature is the fact that “pure” MBL mass and activity has been historically difficult to measure due to interference and cross-talk in assays from other complement activation pathways. As an example, it was shown that standard MBL assays relying on a hemolytic method have functional interference from C1q, and that in order to overcome the interference and get a true measure of MBL amount, anti-C1q antibodies have to be added to overcome the interference (see Herpers, B. et al., 2009, which is hereby incorporated by reference in its entirety). Thus, studies that measured MBL using assays that did not inhibit classical complement pathway protein activity may have failed to detect many cases of MBL deficiency, potentially influencing the outcome of their studies.

In one embodiment, the invention provides a method that employs a high ionic strength buffer to measure only MBL activity and at the same time, inhibits the activity of other complement proteins (e.g., C1q, see Petersen, S. et. al., 2001, which is hereby incorporated by reference in its entirety).

While MBL is made in the liver, it is regarded as an acute-phase protein because the amount produced may increase due to inflammation. Some studies have shown that MBL amount and activity in the plasma can be remarkably consistent over time; repeated measurements in the same patient over a time span of 15-20 years show a very high correlation of MBL concentrations, and are far less variable than lipids or blood pressure. Also, MBL amount and activity display no diurnal variation and are independent of renal function (see Terai, I. et al., 1993, which is hereby incorporated by reference in its entirety). Some studies have suggested that changes in MBL levels during acute phase response are very small when compared with changes in acute phase proteins like CRP (see Hansen, T. K. et al., 2006 and Hansen, T. K. et al., 2003, both of which are hereby incorporated by reference in their entirety). A few studies have shown increases in MBL levels following surgeries and ischemia-reperfusion injury (see Walsh, M. C. et al., 2005, which is hereby incorporated by reference in its entirety) and it has been postulated that this may be due to tissue trauma and inflammation. Therefore, the MBL amount and/or MBL activity, and a derivative index value from both measurements when measured in a healthy patient would be an excellent candidate test for “lifetime” risk prognosis of development of cardiodiabetes, and could identify patients who are as yet asymptomatic so that they could be targeted for aggressive early intervention to prevent development of cardiodiabetic diseases.

Measurement of MBL amount, or activity, may not be sufficient information to gauge risk of cardiovascular disease and cardiodiabetes since both the amount and the functionality can vary greatly between individuals, and there are other factors that are known to contribute significantly to risk. A complete screening approach that encompasses screening for absolute amount of MBL present in serum, and the biological activity level of this protein, in addition to MBL genotype including its promoter region, (see Kuipers, S. et al., 2002, which is hereby incorporated by reference in its entirety) assists in determining which patients have clinically relevant MBL deficiency to enable identification and administration of the most appropriate therapy before cardiodiabetes develops. MBL mass may be combined with activity or an index derived therefrom with additional biomarkers comprising comprehensive diabetic risk status (such as glycemic control, beta cell dysfunction and insulin resistance) to calculate an inclusive MBL index score for ascertaining relative cardiodiabetic risk. Treatment for MBL deficiency exists; intravenous enzyme replacement therapies have been developed. Enzon Pharmaceutical has developed rhMBL and it has been used clinically for treatment of a number of different conditions related to MBL deficiency (see Petersen, K. A. et al., 2006, which is hereby incorporated by reference in its entirety). An MBL derivative, recombinant chimeric lectin 4 (RCL4) is efficient at activating the lectin complement pathway without significant promotion of thrombin-like activity (see Chang, W. C. et al., 2011, which is hereby incorporated by reference in its entirety), and RCL4 and other recombinant chimeric lectin compounds in development hold promise as treatments for MBL deficiency. Additionally, it may be possible to treat all other contributing factors to cardiodiabetes on different physiological axis than MBL itself. As an example, a patient with low MBL mass and activity may be advised that their risk of cardiodiabetes is high due to their index score, but that the risk may be ameliorated by proper diet, exercise, taking a statin, an anti-coagulant, etc. Thus, abnormal MBL may be taken as a risk factor in as much the way Lp(a) is; Lp(a) is a lipoprotein that is highly atherogenic, largely genetic, not subject to diurnal/lifetime variation, and not much affected by therapies available today. Yet, Lp(a) is measured because it may provide clues as to the patient's inherent risk of cardiodiabetic disease, which can, in turn, minimize all other controllable risk factors in an effort to offset the high risk of cardiodiabetic disease conferred by high Lp(a).

The pathophysiology of MBL is complicated; while sufficient MBL is beneficial and limits tissue injury during infections, it appears to mediate tissue injury in other inflammatory states. But because MBL plays a central role in hemostasis, immunity and inflammation, both insufficient and excessive levels of MBL may result in dysregulation of the system and thus increased risk. The previous discussion has been primarily focused on MBL deficient phenotypes and the increased risk of cardiodiabetes and infections/immunodeficiencies. However, excessively high levels of MBL have been implicated in cardiovascular morbidity and mortality, particularly in the context of autoimmune disease. For example, patients with rheumatoid arthritis have higher risk of atherosclerosis and cardiovascular disease that may not be attributable to traditional risk factors. In one study of Danish patients with Rheumatoid Arthritis, high MBL production significantly increased the overall risk of death and cardiovascular death in particular during the course of the study (median follow-up of ten years) (see Troelsen, L. N. et al., 2010, which is hereby incorporated by reference in its entirety). In another cross-sectional study, the MBL-2 genotypes, and serum concentrations of MBL were measured, and compared to the patients' intima-media thickness of the common carotid artery (ccIMT), which measures for subclinical CVD. The ccIMT was related to the serum MBL not linearly, but quadratically. In other words, there was a U-shaped curve wherein deficiency or overabundance of MBL was highly correlated with ccIMT (see Troelsen, L. N. et al., 2010, which is hereby incorporated by reference in its entirety). The investigated MBL genotypes did not correlate.

Many patients with Systemic Lupus Erythematosus (SLE) have significant cardiovascular disease as a complication. Variant alleles of MBL gene are associated with SLE, and severe atherosclerosis. Also, among patients with SLE, those who are homozygous for the O/O genotype develop arterial thrombosis at a very high rate (hazard ratio=7) compared to those with other MBL genotypes (see Ohlenschlaeger, T., 2004, which is hereby incorporated by reference in its entirety). Another study of SLE patients found that the prevalence of cardiovascular disease in the patients with MBL-deficient genotypes was 3.3 times higher than in patients with non-deficient genotypes (see Font, J., 2007, which is hereby incorporated by reference in its entirety).

Thus, MBL may have a role in mediating complications due to ischemia-reperfusion injury. Studies have shown that MBL-null mice have significantly less tissue damage from ischemia-reperfusion injuries in the heart, gut and kidneys. It is known that MBL is deposited on damaged myocardium and activates the complement cascade, leading to tissue injury. High levels of MBL may thus increase the risk of inflammatory damage after ischemia/reperfusion. One study showed that administration of a downstream complement cascade C5 inhibitor reduced mortality after percutaneous coronary intervention. It has been shown that administration of pexelizumab, a monoclonal inhibitor of C5, reduces the risk of death in patients undergoing coronary artery bypass grafting (see Testa, L. et al., 2008, which is hereby incorporated by reference in its entirety). Yet in another study, high plasma MBL and low plasma sC5b-9 were independently associated with increased risk of cardiac dysfunction in STEMI patients treated with pPCI (see Haarh-Pedersen, S. et al., 2009, which is hereby incorporated by reference in its entirety).

MBL-initiated inflammation and complement activation have been implicated in the pathological process of development of T1DM and vascular complications from diabetes. High MBL concentration and high levels of activity have been shown at the time of clinical manifestation of T1DM in juveniles (Bouwman, L. H. et al., 2005, which is hereby incorporated by reference in its entirety). A longitudinal study of 326 Danish patients with T2DM found that the risk of death was significantly higher amount individuals with high levels of MBL (above 1000 μg/L), and added to the predictive power of high CRP. T2DM patients in this study with high MBL levels who did not have albumin in their urine at baseline developed micro- and macro-albuminuria at significantly higher rates than those with low MBL (Hansen, T. K. et al., 2006, which is hereby incorporated by reference in its entirety), indicating a role for MBL in the development of kidney damage from microvascular disease well-documented in T2DM patients. High levels of circulating MBL and genotypes associated with higher amounts of MBL have also been correlated with diabetic nephropathy and cardiovascular disease, in T1DM patients (Hansen, T. K. et al., 2004, Hovind, P. et al., 2005, both of which are hereby incorporated by reference in their entirety). Only ⅓ of patients with diabetes develop nephropathy and/or consequential ESRD. Both higher levels of MBL in the serum and high complex activity have been observed in T1DM patients and patients with diabetic nephropathy, leading to speculation that MBL may be involved by accelerating pathogenesis of the conditions (Ichinose, K. et al., 2007, which is hereby incorporated by reference in its entirety).

The terms “quantities,” “levels,” “amounts,” “concentrations,” and “numbers” when used to describe the amount of various analytes or biomarkers including lipoprotein particles, cholesterol, phospholipid, etc. are herein interchangeable. The term “mass” or “concentration” and “amount” or “level” may be used interchangeably when referring to the absolute measured amount of MBL protein or MBL/MASPs complex contained in a given amount of biological material (e.g. serum or plasma). The term “activity” refers to not the detectable amount, but rather the measurable biological function of mass contained within the given amount, for example, the amount of a complement fragment produced by the MBL/MASP2 complex mass present in a given quantity of plasma is a functional measure of MBL/MASP-2 activity. The terms “index score,” “index value” and “activity index” are interchangeable and mean a number which is part of a range of numbers determined by a mathematical operation performed upon the absolute values of the amount of the MBL measured, and the activity of the MBL measured, in the same sample. The mathematical operation may involve multiplication, division, logarithmic transformation, raising to a power, or any combination thereof. The index value may be compared to the range of index values derived from the experiments described herein in order to determine whether that value correlates with reduced, average or higher risk of cardiodiabetic complications or risk of development of cardiodiabetes. The index value from any given subject or subjects may be compared to index values derived from other empirical studies in which both MBL mass and activity are measured, provided that the index value is calculated in the same manner as the range of index values to which it is being compared for the purpose of risk stratification and provided that the same method of measurement of mass and activity are used in both instances.

“Cardiodiabetes” is defined as any condition related to the development and initiation of the diabetic disease process or cardiovascular disease, or complications arising therefrom, including but not limited to the following: insulin resistance, metabolic syndrome, type 2 diabetes mellitus (T2DM), type 1 diabetes mellitus (T1DM), fatty liver, diabetic nephropathy, diabetic neuropathy, vasculitis, atherosclerosis, coronary artery disease (CAD), arterial thrombosis, ccIMT, vulnerable plaque formation, myocardial infarction (MI), heart failure, cardiomyopathy, endothelial dysfunction, hypertension, occlusive stroke, ischemic stroke, transient ischemic event (TIA), deep vein thrombosis (DVT), dyslipidemia, gestational diabetes (GDM), periodontal disease, obesity, morbid obesity, chronic and acute infections, DIC, pre-term labor, diabetic retinopathy, and systemic or organ-specific inflammation.

The term “subject” as used herein includes, without limitation, mammals, such as humans or non-human animals. Non-human animals may include non-human primates, farm animals, sports animals, rodents or pets. A typical subject is human and may be referred to as a patient. Mammals other than humans can be advantageously used as subjects that represent animal models of the cardiovascular disease or for veterinarian applications.

A “biological sample” encompasses a variety of sample types obtained from a subject with a biological origin. Examples of biological fluid sample include, but are not limited to, blood, cerebral spinal fluid (CSF), interstitial fluid, urine, sputum, saliva, mucous, stool, lymphatic, or any other secretion, excretion, or and other bodily liquid samples. Exemplary biological fluid sample can be a blood component such as plasma, serum, red blood cells, whole blood, platelets, white blood cells, or components or mixtures thereof.

A therapy regimen includes, for example, drugs or supplements. The drug or supplement may be any suitable drug or supplement useful for the treatment or prevention of diabetes and related cardiovascular disease. Examples of suitable agents include an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a PCSK9 inhibitor, an MTP inhibitor, mipmercin, a calcium channel blocker, a beta-adrenergic receptor blocker, an angiotensin system inhibitor, a recombinant chimeric lectin, a complement cascade inhibitor, a complement protein-specific monoclonal antibody, a complement specific antagonist, a serine protease inhibitor, a glitazone, a GLP-1 analog, thiazolidinedionones, biguanides, neglitinides, alpha glucosidase inhibitors, an insulin, a dipeptidyl peptidase IV inhibitor, metformin, a sulfonurea, peptidyl diabetic drugs such as pramlintide and exenatide, or combinations thereof. The agent is administered in an amount effective to treat the cardiovascular disease or disorder or to lower the risk of the subject developing a future cardiovascular disease or disorder.

A therapy regimen may also include treatment for chronic infections such as UTIs, reproductive tract infections, and periodontal disease. Therapies may include appropriate antibiotics and/or other drugs, and surgical procedures and/or dentifrice for the treatment of periodontal disease.

A therapy regimen may include referral to a healthcare specialist or related specialist based on the determining of risk levels. The determining may cause referral to a cardiologist, endocrinologist, ophthalmologist, lipidologist, weight loss specialist, registered dietician, “health coach,” personal trainer, etc. Further therapeutic intervention by specialists based on the determining may take the form of cardiac catherization, stents, imaging, coronary bypass surgeries, EKG, Doppler, hormone testing and adjustments, weight loss regimens, changes in exercise routine, diet, and other personal lifestyle habits.

Anti-inflammatory agents include but are not limited to, Aldlofenac; Aldlometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic and/or fibrinolytic agents include but are not limited to, Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant); rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans; Heparin.

Anti-platelet agents include but are not limited to, Clopridogrel; Sulfinpyrazone; Aspirin; Dipyridamole; Clofibrate; Pyridinol Carbamate; PGE; Glucagon; Antiserotonin drugs; Caffeine; Theophyllin; Pentoxifyllin; Ticlopidine; Anagrelide.

Lipid-reducing agents include but are not limited to, gemfibrozil, cholystyramine, colestipol, nicotinic acid, probucol lovastatin, fluvastatin, simvastatin, atorvastatin, pravastatin, cerivastatin, and other HMG-CoA reductase inhibitors.

Direct thrombin inhibitors include, but are not limited to, hirudin, hirugen, hirulog, agatroban, PPACK, thrombin aptamers.

Glycoprotein IIb/IIIa receptor inhibitors are both antibodies and non-antibodies, and include, but are not limited to, ReoPro (abcixamab), lamifiban, tirofiban.

Calcium channel blockers are a chemically diverse class of compounds having important therapeutic value in the control of a variety of diseases including several cardiovascular disorders, such as hypertension, angina, and cardiac arrhythmias. Calcium channel blockers are a heterogenous group of drugs that prevent or slow the entry of calcium into cells by regulating cellular calcium channels (REMINGTON, THE SCIENCE AND PRACTICE OF PHARMACY (Twenty-First Edition, Mack Publishing Company, 2005), which is hereby incorporated by reference in its entirety). Most of the currently available calcium channel blockers belong to one of three major chemical groups of drugs, the dihydropyridines, such as nifedipine, the phenyl alkyl amines, such as verapamil, and the benzothiazepines, such as diltiazem. Other calcium channel blockers include, but are not limited to, anrinone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and the like and/or pharmaceutically acceptable salts thereof.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, beftunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitril HCl, 1-butylamino-3-(2,5-dichlorophenoxy-)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

An angiotensin system inhibitor is an agent that interferes with the function, synthesis or catabolism of angiotensin II. These agents include, but are not limited to, angiotensin-converting enzyme (“ACE”) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na+ in plasma tend to activate the system, while factors that increase these parameters tend to suppress its function.

Angiotensin (renin-angiotensin) system inhibitors are compounds that act to interfere with the production of angiotensin II from angiotensinogen or angiotensin I or interfere with the activity of angiotensin II. Such inhibitors include compounds that act to inhibit the enzymes involved in the ultimate production of angiotensin II, including renin and ACE. They also include compounds that interfere with the activity of angiotensin II, once produced. Examples of classes of such compounds may include antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists, which will be known to those of skill in the art.

Examples of drugs that act to interfere with PSK9's interaction with LDL receptors includes Aln-PCS (Alnylam); REG 727 (Regeneron); and AMG-145 (Amgen).

The drugs and/or supplements (i.e., therapeutic agents) can be administered via any standard route of administration known in the art, including, but not limited to, parenteral (e.g., intravenous, intraarterial, intramuscular, subcutaneous injection, intrathecal), oral (e.g., dietary), topical, transmucosal, or by inhalation (e.g., intrabronchial, intranasal or oral inhalation, intranasal drops). Typically, oral administration is the preferred mode of administration.

A therapy regimen may also include giving recommendations on making or maintaining lifestyle choices useful for the treatment or prevention of diabetes and cardiovascular disease based on the results of determining the amounts of analytes and calculated scores and their associated risk levels in the subject. The lifestyle choices can involve changes in diet, changes in exercise, reducing or eliminating smoking, or a combination thereof. For example, the therapy regimen may include glucose control, lipid metabolism control, weight loss control, and smoking cessation. As will be understood, the lifestyle choice is one that will affect risk for developing or having a cardiovascular disease or disorder (see Haskell, W. L. et al., 1994; Ornish, D. et al., 1998; and Wister, A. et al., 2007, all of which are hereby incorporated by reference in their entirety).

Reports based on the results of determining the subject's diabetes and related cardiovascular disease risk may be generated. The reports may include suggested therapy regimens selected based on the subject's diabetes and cardiovascular disease risk. This report may be transmitted or distributed to a patient's doctor or directly to the patient. Following transmission or distribution of the report, the subject may be coached or counseled based on the therapy recommendations.

Methods according to the invention may also involve administering the selected therapy regimen to the subject. Accordingly, the invention also relates to methods of treating a subject to reduce the risk of a cardiovascular disease or disorder.

Treating the subject involves administering to the subject an agent suitable to treat a diabetes, or cardiovascular disease or disorder or to lower the risk of a subject developing a future diabetes or cardiovascular disease or disorder. Suitable agents include an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a PCSK9 inhibitor, an MTP inhibitor, mipmercin, a calcium channel blocker, a beta-adrenergic receptor blocker, an angiotensin system inhibitor, a glitazone, a GLP-1 analog, thiazolidinedionones, biguanides, neglitinides, alpha glucosidase inhibitors, an insulin, a dipeptidyl peptidase IV inhibitor, metformin, a sulfonurea, peptidyl diabetic drugs such as pramlintide and exenatide, or combinations thereof. The agent is administered in an amount effective to treat the cardiovascular disease or disorder or to lower the risk of the subject developing a future cardiovascular disease or disorder.

A therapy regimen may also include treatment for chronic infections such as UTIs, reproductive tract infections, and periodontal disease. Therapies may include appropriate antibiotics and/or other drugs, and surgical procedures and/or dentifrice for the treatment of periodontal disease.

A therapy regimen may include referral to a healthcare specialist or related specialist based on the determining of risk levels. The determining may cause referral to a cardiologist, endocrinologist, ophthalmologist, lipidologist, weight loss specialist, registered dietician, “health coach”, personal trainer, or other health services provider. Further therapeutic intervention by specialists based on the determining may take the form of cardiac catherization, stents, imaging, coronary bypass surgeries, EKG, Doppler, hormone testing and adjustments, weight loss regimens, changes in exercise routine, diet, and other personal lifestyle habits.

Monitoring can also assess the risk for developing diabetes and cardiovascular disease. This method involves determining if the subject is at an elevated risk for developing diabetes and cardiovascular disease, which may include assigning the subject to a risk category selected from the group consisting of high risk, intermediate risk, and low risk (i.e., optimal) groups for developing or having diabetes or cardiovascular disease. This method also involves repeating the determining if the subject is at an elevated risk for developing diabetes and cardiovascular disease after a period of time (e.g., before and after therapy). The method may also involve comparing the first and second risk categories determining, based on the comparison, if the subject's risk for developing diabetes and cardiovascular disease has increased or decreased, thereby monitoring the risk for developing diabetes and cardiovascular disease.

The invention herein relates to a comprehensive panel or method that includes the measuring the value of MBL mass (amount or concentration) and/or activity for determination of cardiovascular and cardiodiabetes risk level and therapy guidance. Tests are available to measure the amount, or the activity of MBL based on various parameters, or the genotypes of the MBL coding sequence and/or the promoter sequence (for more details, see Background section). An MBL inclusive Index Value or Score based on combining the measurement values of MBL mass and, optionally, MBL activity, especially in conjunction with other known biomarkers of cardiovascular risk for further risk stratification and therapy guidance.

In one embodiment, a patient sample is contacted and the sample can be tested using known laboratory methods to 1) quantify amount of MBL (MBL mass) present, 2) measure activity of that MBL, and 3) combine the information into a calculated index MBL Activity Score. There are numerous assays in existence to quantify MBL (e.g. ELISAs, electrophoresis) and many ways to assess relative activity (e.g., complement assays).

In one embodiment, the MBL mass can be measured by enzyme-linked immunosorbent assay (ELISA), electrophoresis, double-enzyme immunoassay, immunofluorometry, and/or hemolytic assay.

In another embodiment, the MBL activity level can be measured by ELISA, complement assay and/or mannan capture method assay or by one or more techniques selected from the group consisting of hemolysis assay, mannan capture assay, micro-organism lysis assay, an assay measuring ability to promote opsonization of a particle or micro-organism, and an assay measuring the production of complement components C4b and/or C3b.

Measurements and calculated indices are compared to reference values from a population, standard values derived from the literature and/or from empirical clinical studies. The value representing the measured amount of MBL will be multiplied by a value representing the activity of MBL with optionally other mathematical operations executed on the resulting value to generate an MBL-Inclusive Index Score.

In one embodiment, the absolute value of measured MBL mass is divided by the absolute value of measured MBL activity (i.e. multiplied by the inverse of the measured value of MBL activity), taking the log of that resulting number, and designating that mathematical result as the calculated index value of MBL Activity Score or MBL-Inclusive score. The Index value may be reported as calculated (i.e., a range of real numbers both positive and negative) or the range of real numbers and patient index score may be reported by converting the value to a percentage range.

In another embodiment, the method for predicting susceptibility or likelihood of a subject having a clinically-relevant mannose-binding lectin (MBL) deficiency to develop cardiodiabetes may include obtaining measurement values of MBL mass and MBL activity level; obtaining measurement values for at least one other biomarker, e.g., Fructosamine, C-peptide, and 1, 5 AG; calculating an MBL-inclusive index score based the measurements obtained in steps (a) and (b) using the following equation:

$$LN [ \frac{MBL mass * 1 , 5 {AG}^{1.91}}{MBL activity * {Fructosamine}^{10.67} * C \text{-} {peptide}^{2.29}} ] ;$$

and comparing the MBL-inclusive index to reference values from a population, wherein an elevated MBL-inclusive index score correlates with a range in a higher unit of an ordered distribution of the population and indicates that the subject is less susceptible to or has a less likelihood of developing cardiovascular disease and/or cardiodiabetes, and wherein a low MBL-inclusive index score correlates with a range in a lower unit of an ordered distribution of the population and indicates that the subject is more susceptible to or has an increased likelihood of developing cardiovascular disease and/or cardiodiabetes.

Since too little MBL can be harmful and may increase cardiovascular and other risks, and too much has been associated with risks such as increased arterial intimal thickness in the context of autoimmune disease, there is a U-shaped (quadratic) curve for normal vs. abnormally low (left) and abnormally high (right) MBL amounts, and activities. Thus, the true shape of the range of Index values can also be quadratic, wherein the low and high values of the index range also correspond to increased risk (compared to “normal” values in the middle) for cardiodiabetic disease risk. The index score test's cutoff limits corresponding to risk levels may, therefore, be designated as low-risk in the middle (approximately 50% of the population falling into this category), optionally intermediate risk to the right and/or left of the low-risk and highest risk on the extremes (for example, the top 10% and the bottom 10%, or other partitioned percentages (tertiles, quartiles, quintiles, etc.) empirically determined to correspond best with the risk levels for cardiodiabetic clinical endpoints in a population. Additionally, at least one optional biomarker or test from each of the following groups may be added to the MBL-Inclusive Index Score: biomarkers for inflammation, lipids, biomarkers of cholesterol synthesis, biomarkers of cholesterol absorption, biomarkers of auto-immune conditions, glycemic control, beta cell dysfunction, and insulin resistance. The method can be used to determine which patients have truly elevated risk levels overall and for specific types of cardiovascular and cardiodiabetic adverse events in light of their MBL-Inclusive Index Score. An MBL-Inclusive index score may include any of the biomarkers, measurements, or transformations described in U.S. patent application Ser. No. 14/038,698 and PCT/US13/69257 for predicting risk of cardiodiabetes. Therapies based on the MBL-Inclusive Index Score and optional panel tests may include as examples infusion of recombinant MBL, infusion of an MBL analog and/or derivative, aggressive management of LDL and Apo-B with drugs such as statins and PCSK9 inhibitors, diet and lifestyle intervention, anti-infectives including antibiotics and anti-virals, immunosuppressive therapies, therapies that affect the complement cascade, therapies with compounds designed to mimic one or more biological effects of MBL, and other drug-based and lifestyle-based therapeutic interventions.

Genetic testing for standard known mutations may or may not be included. Genetic testing for other diseases that would contribute to the pathology of aggressive cardiovascular disease such as ApoE genotype and Familial Hypercholesterolemia may also be included.

More accurate determination of which patients require clinical intervention to ameliorate or reduce their risk of cardiovascular and cardiodiabetic morbidity and mortality as a result of their MBL status. Test for MBL-Inclusive Index Score can in many circumstances be done once because there is so little variability through the years and over a person's lifetime. Studies have shown that repeated measurements over a time span of 15 to 20 years show a very high correlation of MBL concentrations, exceeding the long-term consistency of known risk markers such as total serum cholesterol and systolic and diastolic blood pressure. Concentrations of MBL show no diurnal variation and are independent of renal function, and the variations in MBL levels during acute phase responses are very small compared with the changes seen with CRP. (2006 paper from Masako, need reference). The MBL-Inclusive Index Score can be part of a permanent medical record and taken into account for the life of that individual when making decisions regarding treatment due to concomitant risk factors. As such, the MBL-Inclusive Index Score would enable pro-active preventive measures to be taken in high-risk individuals early in life and reduce morbidity and mortality from cardiovascular disease as well as other complications. Since other studies have indicated that MBL levels secreted by the liver into the blood may rise in response to serious injury, inflammation or infection that would initiate an acute phase response, the MBL activity index value may be assessed multiple times, and optionally a comparison may be made between Index values determined in “baseline” samples when a patient is well, and the determinations when a patient is ill, in order to ascertain if the MBL Index indicates the biological response is insufficient, adequate, or excessive; in this instance of repeated measurement the Index value would inform risk classification and guide therapy depending on the specific disease or condition being monitored and/or treated.

### EXAMPLES

### Clinical Study Protocol Study Number 1

All laboratory measurements were performed at Health Diagnostic Laboratory, Inc. (HDL). Of the 217 study participants, there was enough excess sample to determine MBL mass and MBL activity in 195 patients. MBL Mass (amount) was determined using the Hycult Biotech ELISA, MBLHK 323-2. MBL Activity was determined using the Hycult Biotech ELISA HK327 human MBL/MASP-2 Assay. MBL activity was measured via functional MBL/MASP-2 assay because the ability of the MBL/MASP-2 complex to initiate C4 cleavage when it is bound to mannan has been well characterized. This method of measurement was selected because any influence of the classical pathway of complement activation was eliminated by a binding buffer that inhibits the binding of C1q to immune complexes and disruption of the C1 complexes while leaving the natural binding activity of MBL and integrity of MBL complexes intact.

Glucose tolerance testing was performed according to standardized protocol. Fasting blood samples were collected before administration of glucola (75 mg glucose solution), which was consumed within 5 minutes. Additional blood samples were collected at either (1) 30, 60, 90, and 120 minutes, or at (2) 60 and 120 minutes, from completion of the glucola. All patients avoided eating, drinking, or smoking during the testing period.

Subjects: 217 consecutive subjects who had not been diagnosed with diabetes, but who had risk factors detailed below, underwent a 75 g oral glucose tolerance test (OGTT) and fasting blood collection to evaluate risk of diabetes between March 2012 and May 2013 at several outpatient centers across the US (Madison, Wis.; Jackson, Miss.; Montgomery, Ala.; Charleston, S.C.; Seattle, Wash.; and Salt Lake City, Utah). Clinical indications for testing may include obesity, history of first-degree family members with diabetes, and presence of one or more components of the metabolic syndrome, including impaired fasting glucose. Patients who tested positive for Anti-GAD autoantibody were excluded from this analysis. Samples were sent by overnight courier to Health Diagnostic Laboratory, Inc. (Richmond, Va.) for measurement of glucose, insulin, metabolites, and other biomarkers. Subjects with detectable anti-GAD antibody (titer>5 IU/ml) were excluded from this study regardless of T1DM or LADA status. The study protocol was approved by Copernicus Group IRB (NC). All analyses involved de-identified data only and were covered by a waiver of consent and authorization requirements. Insulin resistance (IR) was defined by one or more of the following conditions: fasting glucose≧100 mg/dL, 2-hour glucose≧140 mg/dL, HbA1c≧5.7%, fasting insulin≧12 μU/mL. Transient hyperglycemia (TH) was defined as 30, 60, or 90-minute glucose≧140 mg/dL during OGTT.

### Statistical Methods

All statistical tests were performed with either StatView version 5 or SAS software (version 9.3; SAS Institute). Statistical significance was defined as p<0.05. The results generated via the described statistical methods were further analyzed for the utility of all biomarkers measured and enumerated in this patent application to identify and classify patients who were at risk of cardiodiabetes.

The following cardiodiabetes clinical endpoints were dependent variables in logistic regression models: 1-hour glucose≧155 mg/dL, 2-hour glucose≧140 mg/dL, pre-diabetes and diabetes by ADA guidelines. Mannose Binding Lectin (MBL) mass and activity, their product and quotient were evaluated as predictor variables; these included their raw values and various non-linear transformations, i.e. natural logarithm, square-root, and quadratic. Pearson and Spearman Rank correlations were tested between the continuous endpoints 1-hour and 2-hour glucose and the MBL metrics. The models were adjusted for age, gender, and BMI.

Next, the following list of biomarkers were added to the multivariable logistic regression models: Fructosamine, Mannose, 1,5 AG, AHB, Amylase, GLP1, C-peptide/Pro-insulin, C-peptide, Pro-insulin, Leptin, Adiponectin, Ferritin, FFA, OA, LGPC, apoB48, and remnant lipoprotein cholesterol. Various variable selection techniques were used to determine the most predictive set of biomarkers. SAS version 9.3 software was used for all analyses, and a critical level alpha<0.05 was used to prescribe statistical significance.

### Statistical Methods for Clustering Analysis and Corresponding Heat Map

Principal Component Analysis (PC) followed by clustering were used to identify biomarkers included in our panel of claimed analytes that add specific and unique information when used in combination. The analyses presented here are to illustrate that MBL mass and/or MBL activity and/or index scores derived therefrom cluster in such a way as to be their own related axis of information, such that they are additive and synergistic when included with biomarkers from other axis of information in the clinical evaluation of cardiodiabetic risk. The clustering analyses herein are intended as a non-limiting example and does not necessarily exemplify the preferred embodiments of the claims herein.

For the clustering analyses presented and described in Tables 1-7, each disjoint cluster includes a cluster component score based on a linear combination of the weighted, standardized biomarker values contained within that cluster. The linear combinations were obtained using principal components (PC) analysis to maximize the amount of explained variability; however, the PC are rotated (i.e. not orthogonal) hence the disjoint clusters are correlated. PC identifies groups of well-correlated biomarkers (that share an unobserved dimension in the data). The natural log was taken to make the biomarkers more symmetric and thus reduce the influence of outliers in the dataset Inherent in the PC analysis are methods to optimize explained variability, which is the variability that is not random. PC explains total variability which includes common (shared) variability among the markers, and random error. The number of clusters was determined by considering: eigenvalues, minimum R-squared value between a biomarker and its cluster component score, total variability explained in the data, and subject matter knowledge. The clusters biomarkers membership and the amount of variation explained in each biomarker by its own cluster are given in the related Tables. A heat map (FIG. 1) was used to show the absolute value of the correlation between the values of each biomarker and each cluster component score. The clusters form blocks of high correlation values, which can be seen on the main diagonal of the heat map. This indicates those variables that are homogeneous (shown in yellow and light tan color). Whereas blue and purple colors indicate independence between clusters and biomarkers; green represents moderate correlations. To relate the inclusion of biomarkers from groups claimed in this application to improvement of an index risk score, analysis in Table 6 was performed. The area under the OGTT curve for FFA times C-peptide, and 1-hr, and 2-hr glucose responses were modeled as the dependent variables to determine which biomarkers are related to these endpoints; this analysis is a non-limiting example of how meaning is provided and assigned to the clusters. The clustering analyses provide the rationale for adding additional biomarkers to MBL mass, MBL activity, or an index value derived therefrom; measurement of additional biomarkers from other clusters informs the test with pertinent information pertaining to cardiodiabetic status and risk from different axis of physiology. These additional biomarkers therefore further inform risk assessment and diagnosis, prognosis, and method of optimal therapeutic intervention to minimize cardiodiabetic risk.

It should be noted that not all data analyses contain data from the total number of study subjects (217). This is because not all tests were run on all samples due to factors beyond the control of HDL, such as insufficient sample volume to perform specialty tests or errors in collection procedure. Throughout this application the exact number of patients included in each statistical analysis have been noted.

### Results

TABLE 1
Cluster summary for 13 clusters (N = 162); Study #1

| | | Cluster | Variation | Proportion | Second |
|---|---|---|---|---|---|
| Cluster | Members | Variation | Explained | Explained | Eigenvalue |
| 1 | 3 | 3 | 2.814973 | 0.9383 | 0.1744 |
| 2 | 4 | 4 | 2.917765 | 0.7294 | 0.4742 |
| 3 | 3 | 3 | 2.846232 | 0.9487 | 0.1397 |
| 4 | 3 | 3 | 2.17735 | 0.7258 | 0.6496 |
| 5 | 2 | 2 | 1.72955 | 0.8648 | 0.2704 |
| 6 | 2 | 2 | 1.312203 | 0.6561 | 0.6878 |
| 7 | 2 | 2 | 1.76549 | 0.8827 | 0.2345 |
| 8 | 3 | 3 | 1.992144 | 0.6640 | 0.7319 |
| 9 | 1 | 1 | 1 | 1.0000 | |
| 10 | 2 | 2 | 1.302942 | 0.6515 | 0.6971 |
| 11 | 2 | 2 | 1.586604 | 0.7933 | 0.4134 |
| 12 | 1 | 1 | 1 | 1.0000 | |
| 13 | 1 | 1 | 1 | 1.0000 | |

Total variation explained = 23.44525 Proportion = 0.8085

TABLE 2
Biomarker summary for 13 clusters (N = 162); Study
#1. Proportion of explained variability in each
biomarker by its cluster component score (first column,
explained variability with own cluster, R-squared

| | | R-squared with | | |
|---|---|---|---|---|
| | | Own | Next | 1-R**2 |
| Cluster | Variable | Cluster | Closest | Ratio |
| Cluster 1 | ln_leptin | 0.9755 | 0.3697 | 0.0389 |
| | ln_leptin_bmi | 0.9582 | 0.2985 | 0.0596 |
| | ln_leptin_adipo | 0.8813 | 0.4550 | 0.2178 |
| Cluster 2 | ln_{—}rlpch | 0.7904 | 0.1462 | 0.2455 |
| | ln_{—}ldltg | 0.7474 | 0.2127 | 0.3209 |
| | ln_adipo | 0.6438 | 0.1965 | 0.4433 |
| | LP_IR_SCORE | 0.7362 | 0.2827 | 0.3678 |
| Cluster 3 | ln_homa_ir | 0.9739 | 0.3709 | 0.0415 |
| | ln_insulin | 0.9675 | 0.3925 | 0.0535 |
| | ln_cpep | 0.9049 | 0.3488 | 0.1461 |
| Cluster 4 | ln_ffa | 0.8061 | 0.0506 | 0.2043 |
| | ln_ahb | 0.5074 | 0.0599 | 0.5239 |
| | ln_oa | 0.8639 | 0.0485 | 0.1431 |
| Cluster 5 | ln_{—}mbl_{—}masp_{—}2_{—}function | 0.8648 | 0.0353 | 0.1402 |
| | ln_{—}mbl_{—}mass | 0.8648 | 0.0506 | 0.1424 |
| Cluster 6 | GLP_{—}1 | 0.6561 | 0.0876 | 0.3769 |
| | ln_ferr | 0.6561 | 0.0552 | 0.3640 |
| Cluster 7 | ln_proinsulin | 0.8827 | 0.6008 | 0.2937 |
| | ln_proinsulin_cpep | 0.8827 | 0.0953 | 0.1296 |
| Cluster 8 | ln_fruct | 0.6779 | 0.1683 | 0.3872 |
| | ln_lgpc | 0.4822 | 0.1921 | 0.6409 |
| | GGAP | 0.8320 | 0.3015 | 0.2405 |
| Cluster 9 | Glycomark_{—}1_{—}5_{—}AG | 1.0000 | 0.0456 | 0.0000 |
| Cluster 10 | ln_{—}human_{—}mannose | 0.6515 | 0.0488 | 0.3664 |
| | ln_{—}apob_{—}48 | 0.6515 | 0.2245 | 0.4494 |
| Cluster 11 | ln_gluc | 0.7933 | 0.2464 | 0.2743 |
| | ln_alc | 0.7933 | 0.2156 | 0.2635 |
| Cluster 12 | ln_{—}amylase | 1.0000 | 0.1104 | 0.0000 |
| Cluster 13 | ln_{—}cd_{—}26 | 1.0000 | 0.0535 | 0.0000 |

Newly added 10 biomarkers (beyond 7 cluster model) in bold.

TABLE 3
Table 3. Comparison of sets of biomarkers and OGTT endpoints (N =
188); The OGTT Index (see U.S. Provisional patent application No.
61/847,922, filed Jul. 17, 2013, which is hereby incorporated by
reference in its entirety) was calculated for all subjects, and then
it plus the 10 additional biomarkers listed in this table were
eligible to be selected as predictor variables in linear models for
the dependent responses (i.e. endpoints). To improve generalization
of the results, 1000 bootstrapped samples were created and predictor
variables were selected if they were included in the final model
that minimized Akaike's information criterion (AIC) in at least 500
of the samples. Mannose Binding Lectin (MBL) mass and 1,5 AG
independently improved prediction of the OGTT endpoints. MBL
functional activity (MBL/MASP-2) was also selected in over 50% of
the models for the product of C-peptide AUC and FFA AUC; it is
shown in the same dimension as MBL mass in the cluster analyses.
Amylase was also selected, which is its own dimension of information.

| | Endpoints | | | | |
|---|---|---|---|---|---|
| | Ln(C- | 1-hr | 2-hr | 1-hr | 2-hr |
| | peptide | Glucose | Glucose | Glucose ≧ | Glucose ≧ |
| | AUC | Contin- | Contin- | 155 | 140 |
| | * FFA AUC) | uous | uous | mg/dL | mg/dL |
| OGTT Index | X | X | X | X | X |
| Ln(functional | X | | | | |
| MBL/MASP-2) | | | | | |
| Ln(MBL mass) | X | X | X | X | X |
| Ln(Amylase) | X | | | | |
| GLP-1 | | | | | |
| Ln(Mannose) | | | | | |
| 1,5 AG | | X | X | X | X |
| Ln(LDL-TG) | | | | | |
| Ln(Remnant | | | | | |
| Lipoprotein-C) | | | | | |
| Ln(ApoB48) | | | | | |
| Ln(CD26) | | | | | |

X = indicates a variable was selected in at least 500 of the 1000 bootstrapped samples.

TABLE 4
Cluster Summary for 11 cluster analysis N = 164, P = 25
Cluster Summary for 11 Clusters

| | | Cluster | Variation | Proportion | Second |
|---|---|---|---|---|---|
| Cluster | Members | Variation | Explained | Explained | Eigenvalue |
| 1 | 4 | 4 | 2.590436 | 0.6476 | 0.7072 |
| 2 | 3 | 3 | 2.366879 | 0.7890 | 0.6286 |
| 3 | 3 | 3 | 2.180443 | 0.7268 | 0.6588 |
| 4 | 3 | 3 | 2.056721 | 0.6856 | 0.6900 |
| 5 | 3 | 3 | 1.900259 | 0.6334 | 0.7371 |
| 6 | 2 | 2 | 1.731315 | 0.8657 | 0.2687 |
| 7 | 2 | 2 | 1.306002 | 0.6530 | 0.6940 |
| 8 | 1 | 1 | 1 | 1.0000 | |
| 9 | 1 | 1 | 1 | 1.0000 | |
| 10 | 2 | 2 | 1.665054 | 0.8325 | 0.3349 |
| 11 | 1 | 1 | 1 | 1.0000 | |

Total variation explained = 18.79711 Proportion = 0.7519

TABLE 5
Biomarker Clusters for 11 cluster analysis N = 164, P = 25

| | | R-squared with | | |
|---|---|---|---|---|
| 11 Clusters | | Own | Next | 1-R**2 |
| Cluster | Variable | Cluster | Closest | Ratio |
| Cluster 1 | LN_GLUC0 | 0.6044 | 0.0748 | 0.4276 |
| | HBA1C | 0.6260 | 0.1808 | 0.4565 |
| | C_PEP0 | 0.6579 | 0.3768 | 0.5489 |
| | LN_PROINSULIN | 0.7021 | 0.2711 | 0.4087 |
| Cluster 2 | CPEP_INSULIN0 | 0.5149 | 0.1547 | 0.5739 |
| | LN_PRO_INSULIN0 | 0.8943 | 0.0298 | 0.1089 |
| | LN_CPEPPRO_INSULIN0 | 0.9577 | 0.0420 | 0.0441 |
| Cluster 3 | LN_AHB | 0.4987 | 0.0514 | 0.5285 |
| | FFA | 0.8091 | 0.0288 | 0.1965 |
| | oa_num | 0.8726 | 0.0342 | 0.1319 |
| Cluster 4 | Leptin | 0.7840 | 0.2100 | 0.2735 |
| | LGPC | 0.4677 | 0.1501 | 0.6263 |
| | BMI | 0.8050 | 0.3488 | 0.2994 |
| Cluster 5 | LN_ADIPONECTIN | 0.6321 | 0.1342 | 0.4249 |
| | LN_APOB48 | 0.4866 | 0.0938 | 0.5666 |
| | LN_RLP_C | 0.7816 | 0.0789 | 0.2371 |
| Cluster 6 | LN_MLB_MASS | 0.8657 | 0.0599 | 0.1429 |
| | LN_MLB_MASP2 | 0.8657 | 0.0156 | 0.1365 |
| Cluster 7 | GLP1 | 0.6530 | 0.0935 | 0.3828 |
| | FERR | 0.6530 | 0.0358 | 0.3599 |
| Cluster 8 | AG15 | 1.0000 | 0.0360 | 0.0000 |
| Cluster 9 | LN_MANNOSE | 1.0000 | 0.0547 | 0.0000 |
| Cluster 10 | FRUCT | 0.8325 | 0.1465 | 0.1962 |
| | GGAP | 0.8325 | 0.3901 | 0.2746 |
| Cluster 11 | AMYLASE | 1.0000 | 0.0802 | 0.0000 |

TABLE 6
Cluster Summary for 16 cluster analysis N = 124, P = 43
Cluster Summary for 16 Clusters

| | | Cluster | Variation | Proportion | Second |
|---|---|---|---|---|---|
| Cluster | Members | Variation | Explained | Explained | Eigenvalue |
| 1 | 4 | 4 | 3.07437 | 0.7686 | 0.6052 |
| 2 | 7 | 7 | 5.867464 | 0.8382 | 0.5197 |
| 3 | 4 | 4 | 2.869743 | 0.7174 | 0.5192 |
| 4 | 3 | 3 | 2.282467 | 0.7608 | 0.7122 |
| 5 | 3 | 3 | 2.1976 | 0.7325 | 0.6468 |
| 6 | 4 | 4 | 2.710084 | 0.6775 | 0.6622 |
| 7 | 2 | 2 | 1.73338 | 0.8667 | 0.2666 |
| 8 | 4 | 4 | 3.180882 | 0.7952 | 0.4224 |
| 9 | 2 | 2 | 1.324041 | 0.6620 | 0.6760 |
| 10 | 3 | 3 | 1.972642 | 0.6575 | 0.7059 |
| 11 | 2 | 2 | 1.257473 | 0.6287 | 0.7425 |
| 12 | 1 | 1 | 1 | 1.0000 | |
| 13 | 1 | 1 | 1 | 1.0000 | |
| 14 | 1 | 1 | 1 | 1.0000 | |
| 15 | 1 | 1 | 1 | 1.0000 | |
| 16 | 1 | 1 | 1 | 1.0000 | |

Total variation explained = 33.47015 Proportion = 0.7784

TABLE 7
Biomarker Clusters for 16 cluster analysis N = 124, P = 43

| | | R-squared with | | |
|---|---|---|---|---|
| 16 Clusters | | Own | Next | 1-R**2 |
| Cluster | Variable | Cluster | Closest | Ratio |
| Cluster 1 | LN_ADIPONECTIN | 0.5142 | 0.3151 | 0.7093 |
| | HDL_C | 0.9340 | 0.2913 | 0.0932 |
| | APO_A1 | 0.7855 | 0.1276 | 0.2459 |
| | LN_HDL2 | 0.8407 | 0.3186 | 0.2338 |
| Cluster 2 | LDL_C | 0.8571 | 0.0187 | 0.1456 |
| | LDL_P | 0.8205 | 0.2140 | 0.2284 |
| | TCHOL | 0.7883 | 0.0633 | 0.2260 |
| | N_HDL_C | 0.9556 | 0.1649 | 0.0531 |
| | SDLDL | 0.7539 | 0.4874 | 0.4801 |
| | apo_b_num | 0.9585 | 0.1354 | 0.0479 |
| | APOB_APOA1 | 0.7335 | 0.2821 | 0.3712 |
| Cluster 3 | Leptin | 0.7541 | 0.2702 | 0.3370 |
| | BMI | 0.7614 | 0.3912 | 0.3919 |
| | fibrinc_num | 0.6704 | 0.1548 | 0.3899 |
| | LN_CRP | 0.6838 | 0.2367 | 0.4142 |
| Cluster 4 | CPEP_INSULIN0 | 0.4277 | 0.2132 | 0.7274 |
| | LN_PRO_INSULIN0 | 0.8972 | 0.0475 | 0.1079 |
| | LN_CPEPPRO_INSULIN0 | 0.9576 | 0.0750 | 0.0459 |
| Cluster 5 | LN_AHB | 0.5036 | 0.0557 | 0.5257 |
| | FFA | 0.8290 | 0.0532 | 0.1806 |
| | oa_num | 0.8650 | 0.0901 | 0.1484 |
| Cluster 6 | LN_GLUC0 | 0.6186 | 0.0630 | 0.4070 |
| | HBA1C | 0.6881 | 0.1465 | 0.3654 |
| | C_PEP0 | 0.6870 | 0.3452 | 0.4779 |
| | LN_PROINSULIN | 0.7163 | 0.2173 | 0.3624 |
| Cluster 7 | LN_MLB_MASS | 0.8667 | 0.0276 | 0.1371 |
| | LN_MLB_MASP2 | 0.8667 | 0.0173 | 0.1357 |
| Cluster 8 | LP_IR_SCORE | 0.7365 | 0.5757 | 0.6209 |
| | LN_TRIG | 0.9089 | 0.2359 | 0.1192 |
| | LN_RLP_C | 0.8395 | 0.2611 | 0.2173 |
| | LN_SDLDL_LDL | 0.6960 | 0.1175 | 0.3445 |
| Cluster 9 | GLP1 | 0.6620 | 0.0707 | 0.3637 |
| | FERR | 0.6620 | 0.0578 | 0.3587 |
| Cluster 10 | FRUCT | 0.6591 | 0.1428 | 0.3976 |
| | GGAP | 0.8035 | 0.4071 | 0.3314 |
| | LGPC | 0.5100 | 0.2570 | 0.6595 |
| Cluster 11 | LN_MANNOSE | 0.6287 | 0.0484 | 0.3902 |
| | LN_APOB48 | 0.6287 | 0.1571 | 0.4404 |
| Cluster 12 | AG15 | 1.0000 | 0.0556 | 0.0000 |
| Cluster 13 | LPPLA2 | 1.0000 | 0.1447 | 0.0000 |
| Cluster 14 | AMYLASE | 1.0000 | 0.1219 | 0.0000 |
| Cluster 15 | MPO | 1.0000 | 0.1496 | 0.0000 |
| Cluster 16 | LPA | 1.0000 | 0.0193 | 0.0000 |

The results from study number 1 were further analyzed in order to determine if mathematical transformations of MBL amounts, MBL activity, and indices derived from combining these mathematically, could be correlated with or predictive of certain clinical endpoints and outcomes related to cardiodiabetes risk determination. The study was conducted on subjects who had not been previously diagnosed as diabetic, but who had at least one clinical indication of increased risk of development of diabetes, including obesity, history of first-degree family members with diabetes, and presence of one or more components of the metabolic syndrome, including impaired fasting glucose. The clinical endpoints studied in the apparently normal but at-risk population were existence of diabetic condition (T2DM), existence of pre-diabetes, and abnormally high elevations of blood glucose during an OGTT (1-hr Glucose≧155 mg/dL, 2-hr Glucose≧140 mg/dL) that are well known risk factors for development of T2DM and cardiodiabetic comorbidities.

### Results

Descriptive statistics are provided in Table 8; the natural logarithm transformation made the distribution of raw values more symmetrical for MBL mass, activity, and mass/activity ratio; thereby reducing leverage of extreme values (FIGS. 2-5). There were significant unadjusted correlations (−0.16 to −0.19, p-value<0.05) between 1-hour and 2-hour continuous glucose measures with MBL mass and MBL mass/activity ratio (Tables 9-10, FIG. 6). The correlation between log(mass) and log(2-hour glucose) remained significant (r=−0.15, p-value=0.047) in minimally adjusted models (adjusted for age, gender, and BMI). Log(mass) and log(mass/activity) were significant predictor variables for prevalent diabetes (Table 11). A 1 standard deviation (SD) increase in either of these variables reduced the likelihood of having diabetes by about 50-60%. The linearity assumption was relaxed and tertiles of MBL mass and mass/activity were formed as (<154, 154-459, >459 ng/mL) and (<0.80, 0.80-1.45, >1.45), respectively. Then the middle tertile was set as the reference level, and the odds of having diabetes was calculated for patients in the lowest and highest tertiles. Patients in the lowest tertile of either mass or mass/activity ratio were 3-4 times more likely to have diabetes; however, there were no significant differences between the highest and middle tertiles for any of the endpoints (Table 11). Unadjusted associations are shown in Table 14.

Table 12 shows the significant groups of biomarkers that were selected into the various logistic regression models, which were adjusted for age, gender, and BMI. When predicting prevalence of diabetes MBL mass/activity was a significant predictor variable; along with Fructosamine, C-peptide, and 1,5 AG. An index was created to combine all of these results into a single composite biomarker, which had a generalized r-squared value of 0.52 and fit the data well (Hosmer-Lemeshow p=0.72). The ROC curve AUC was 0.93 (FIG. 6). A plot of the probability for having diabetes versus MBL mass/activity value, while holding the other biomarkers at their mean values, is shown in FIG. 7.

Log(MBL mass) was a useful predictor variable to classify patients with previously unknown status as diabetic, potentially through its correlation with OGTT 2-hour glucose. An ‘index’ comprised of more than one biomarker may include log(MBL mass/activity), which has clinical utility in minimally adjusted models (age, gender, BMI). Adding biomarkers of glycemic control and beta cell stress/dysfunction such as the combination of fructosamine, 1,5 AG, and C-peptide improved the model performance for diabetes prediction compared to the index of log(MBL mass/activity) alone (Table 13, FIG. 7, FIG. 8). Additionally, strong correlation of log(MBL mass/activity) with abnormally high 1 hr glucose in an OGTT, as measured by Pearson correlation coefficient (P=0.052) and Spearman rank correlation coefficients (P=0.028) demonstrate the utility of this index in predicting which patients will have post-prandial hyperglycemia (termed glucose excursions) at 1 hr post OGTT (FIG. 6, tables 9 and 10). Interestingly, the biomarker 1,5 AG is known to indicate clinically significant post-prandial glucose excursions when blood glucose rises to above the renal threshold of 180 mg/dl. This raises the possibility that in a more highly powered study the MBL Index value may add to the predictive value for other biomarkers of post-prandial hyperglycemia. Examples: 1,5 AG and AHB.

Other claimed biomarkers when added to the MBL Index score improved the odds ratio per 1 SD increase in the Index score for various clinical endpoints in minimally adjusted models (Table 12). For high 1 hr glucose, fructosamine, AHB, proinsulin and the lipid biomarker LGPC were significant. For high 2 hr glucose, fructosamine, C-peptide and free fatty acids were significant. For pre-diabetes, mannose, c-peptide and LGPC improved, and as previously mentioned fructosamine, c-peptide and 1,5 AG improved the discriminatory power of the Index Score significantly.

TABLE 8
Descriptive Statistics

| Variable | N | N Miss | Mean | Std Dev | Minimum | Maximum | Skewness | Kurtosis |
|---|---|---|---|---|---|---|---|---|
| Mass | 195 | 0 | 412.46 | 480.94 | 8.66 | 3330.89 | 2.75 | 10.96 |
| Log(Mass) | 195 | 0 | 5.26 | 1.49 | 2.16 | 8.11 | −0.76 | −0.29 |
| Activity | 195 | 0 | 414.58 | 607.01 | 41.16 | 3098.55 | 2.88 | 8.42 |
| Log(Activity) | 195 | 0 | 5.41 | 1.01 | 3.72 | 8.04 | 0.86 | −0.08 |
| Mass/Activity | 195 | 0 | 1.28 | 1.01 | 0.05 | 5.67 | 1.43 | 3.13 |
| Log(Mass/Activity) | 195 | 0 | −0.15 | 1.03 | −3.05 | 1.73 | −0.86 | −0.04 |

Notes:
1) Mass = Mannose Binding Lectin (MBL) Mass
2) Log = natural logarithm
3) Activity = Functional MBL/MASP-2

TABLE 9
Pearson Correlation Coefficients

| | Log(2-hour glucose) | Log(1-hr glucose) |
|---|---|---|
| Log(Mass) | r = −0.19372 | −0.15476 |
| | P-value = 0.0067 | 0.032 |
| Log(Activity) | −0.09524 | −0.08424 |
| | 0.19 | 0.24 |
| Log(Mass/Activity) | −0.18652 | −0.14039 |
| | 0.0090 | 0.052 |

TABLE 10
Spearman Rank Correlation Coefficients

| | Log(2-hour glucose) | Log(1-hr glucose) |
|---|---|---|
| Log(Mass) | rho = −0.16664 | −0.13441 |
| | p-value = 0.020 | 0.062 |
| Log(Activity) | −0.12979 | −0.10698 |
| | 0.071 | 0.14 |
| Log(Mass/Activity) | −0.18038 | −0.15871 |
| | 0.012 | 0.028 |

TABLE 11
Multivariable Adjusted Associations
between MBL and Clinical Outcomes

| Prevalent | | | Log(Mass/ |
|---|---|---|---|
| Outcomes* | Log(Mass) | Log(Activity) | Activity) |
| | Odds Ratios (p-value) per 1 standard deviation increase | | |
| 1-hr glucose ≧ | 0.81 (0.18) | 0.98 (0.89) | 0.75 (0.071) |
| 155 mg/dL | | | |
| (events = 85) | | | |
| 2-hr glucose ≧ | 0.80 (0.16) | 0.91 (0.56) | 0.79 (0.15) |
| 140 mg/dL | | | |
| (events = 55) | | | |
| Prediabetes | 1.29 (0.18) | 1.35 (0.099) | 1.06 (0.77) |
| (events = 62) | | | |
| Diabetes | 0.53 (0.0062) | 0.88 (0.62) | 0.41 (0.0004) |
| (events = 21) | | | |
| | Odds Ratios (p-value) 1^{st} tertile versus 2^{nd} | | |
| | (low vs. medium) | | |
| 1-hr glucose ≧ | 1.11 (0.79) | 1.26 (0.55) | 1.68 (0.17) |
| 155 mg/dL | | | |
| 2-hr glucose ≧ | 1.93 (0.11) | 1.22 (0.61) | 0.91 (0.80) |
| 140 mg/dL | | | |
| Prediabetes | 0.55 (0.18) | 0.84 (0.70) | 0.59 (0.22) |
| Diabetes | 4.09 (0.047) | 2.80 (0.12) | 3.31 (0.046) |
| | Odds Ratios (p-value) 3^{rd} tertile versus 2^{nd} | | |
| | (high vs. medium) | | |
| 1-hr glucose ≧ | 1.01 (0.98) | 1.09 (0.83) | 1.00 (1.00) |
| 155 mg/dL | | | |
| 2-hr glucose ≧ | 1.26 (0.58) | 0.87 (0.74) | 0.55 (0.16) |
| 140 mg/dL | | | |
| Prediabetes | 1.24 (0.60) | 1.46 (0.36) | 0.76 (0.52) |
| Diabetes | 1.30 (0.75) | 1.07 (0.93) | 0.40 (0.31) |

*All models adjusted for age, gender, and BMI.

TABLE 12
Possible groups of biomarkers for an ‘index’
including MBL mass/activity

| | OR (p-value) per 1 SD | |
|---|---|---|
| | increase in Log(Mass/ | Additional Significant |
| Prevalent Outcome | Activity) | Biomarkers |
| 1-hr glucose ≧ 155 | 0.68 (0.10) | Fructosamine, AHB, |
| mg/dL | | Proinsulin, LGPC |
| (events = 70/166) | | |
| 2-hr glucose ≧ 140 | 0.82 (0.30) | Fructosamine, C-peptide, |
| mg/dL | | FFA |
| (events = 45/168) | | |
| Prediabetes | 0.92 (0.73) | Mannose, C-peptide, LGPC |
| (events = 59/146) | | |
| Diabetes | 0.32 (0.0011) | Fructosamine, C-peptide, |
| (events = 18/164) | | 1,5 AG |

OR = odds ratio; All models adjusted for age, gender, and BMI.

TABLE 13
Predict Diabetes, generalized R² = 0.257, max-rescaled R² = 0.519
$Diabetes Index = LN [ \frac{MBL mass * 1 , 5 {AG}^{1.91}}{\begin{matrix}MBL activity * \\ {Fructosamine}^{10.67} * \\ C \text{-} {peptide}^{2.29}\end{matrix}} ]$
Analysis of Maximum Likelihood Estimates

| | | | Stan- | Wald | |
|---|---|---|---|---|---|
| | | | dard | Chi- | Pr > |
| Parameter | DF | Estimate | Error | Square | ChiSq |
| Intercept | 1 | −60.8390 | 17.5920 | 11.9601 | 0.0005 |
| LN(MBL | 1 | −1.0386 | 0.3128 | 11.0216 | 0.0009 |
| mass/ | | | | | |
| activity) | | | | | |
| LN(1,5 AG) | 1 | −1.9805 | 0.6542 | 9.1635 | 0.0025 |
| LN(Fructos- | 1 | 11.0860 | 3.1623 | 12.2895 | 0.0005 |
| amine) | | | | | |
| LN(C- | 1 | 2.3778 | 0.7175 | 10.9823 | 0.0009 |
| peptide) | | | | | |

LN = natural logarithm; MBL Mass [ng/mL];
MBL Activity [U/mL]; 1,5 AG [μg/mL];
Fructosamine [μmol/L]; C-peptide [ng/mL]
Hosmer and Lemeshow Goodness-of-Fit Test

| | | |
|---|---|---|
| Chi-Square | DF | Pr > ChiSq |
| 5.3851 | 8 | 0.7157 |

TABLE 14
Unadjusted Associations between Mannose Binding
Lectin (MBL) and Clinical Outcomes

| Prevalent | | | Log(Mass/ |
|---|---|---|---|
| Outcomes | Log(Mass) | Log(Activity) | Activity) |
| | Odds Ratios (p-value) per 1 standard deviation increase | | |
| 1-hr glucose ≧ | 0.75 (0.054) | 0.94 (0.66) | 0.71 (0.020) |
| 155 mg/dL | | | |
| (events = 85) | | | |
| 2-hr glucose ≧ | 0.76 (0.080) | 0.90 (0.50) | 0.75 (0.062) |
| 140 mg/dL | | | |
| (events = 55) | | | |
| Prediabetes | 1.13 (0.47) | 1.28 (0.15) | 0.93 (0.70) |
| (events = 62) | | | |
| Diabetes | 0.53 (0.0030) | 0.92 (0.72) | 0.40 (<0.0001) |
| (events = 21) | | | |
| | Odds Ratios (p-value) 1^{st} tertile versus 2^{nd} | | |
| | (low vs. medium) | | |
| 1-hr glucose ≧ | 1.29 (0.48) | 1.50 (0.25) | 1.76 (0.11) |
| 155 mg/dL | | | |
| 2-hr glucose ≧ | 2.13 (0.056) | 1.45 (0.34) | 1.00 (1.00) |
| 140 mg/dL | | | |
| Prediabetes | 0.69 (0.38) | 1.11 (0.81) | 0.64 (0.27) |
| Diabetes | 5.27 (0.013) | 3.38 (0.046) | 3.16 (0.039) |
| | Odds Ratios (p-value) 3^{rd} tertile versus 2^{nd} | | |
| | (high vs. medium) | | |
| 1-hr glucose ≧ | 0.97 (0.94) | 1.10 (0.80) | 0.86 (0.67) |
| 155 mg/dL | | | |
| 2-hr glucose ≧ | 1.29 (0.54) | 0.92 (0.84) | 0.52 (0.11) |
| 140 mg/dL | | | |
| Prediabetes | 1.15 (0.72) | 1.59 (0.24) | 0.62 (0.23) |
| Diabetes | 1.73 (0.47) | 1.25 (0.75) | 0.38 (0.26) |

A diagnostic panel made up of tests that 1) quantify amount of MBL present, 2) measure activity of that MBL, and 3) combine the information into a calculated MBL Index Score would be ideal. Optionally, at least one other biomarker of cardiovascular risk such as LDL-P, LDL-C, LDL particle size, ApoE, and Lp(a) as non-limiting examples could be added. Optionally, at least one biomarker of insulin resistance, glycemic control, and/or beta cell dysfunction could be added. Optionally, genotyping could also be added.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

All publications and patent applications mentioned in this specification, including those listed below, are herein incorporated by reference in their entirety to the same extent as if each individual publication or patent application was specifically and individually indicated to be incorporated by reference.
1) Megia, A. et al., Mannose-binding lectin gene polymorphisms are associated with gestational diabetes mellitus, J. Clin. Endocrinol. Metab., 89(10):5081-5087 (2004)
2) Fraser, D. A. and Tenner, A J, Innate immune proteins C1q and mannan-binding lectin enhance clearance of atherogenic lipoproteins by human monocytes and macrophages, J. Immunol., 185(7):3932-3939 (2010)
3) Saevarsdottir, S. et al., Mannan binding lectin as an adjunct to risk assessment for myocardial infarction in individuals with enhanced risk, J. Exp. Med., 201(1):117-125 (2005)
4) Vengen, I. T. et al., Mannose-binding lectin deficiency is associated with myocardial infarction: The HUNT2 study in Norway, PLoS ONE 7(7): e42113 (2012)
5) Chalmers, J. D. et al., Impact of mannose-binding lectin insufficiency on the course of cystic fibrosis: a review and meta-analysis, Glycobiology, 21(3):271-282 (2011).
6) Wallis, R. and Lynch, N. J., Biochemistry and genetics of the collectins, In: Kilpatrick, D., editor, Collagen-Related Lectins in Innate Immunity, Trivandrum: Research Signpost, Kerela, India, 2007, pp. 33-56
7) Swierzko, A. S. et al., Mannan-binding lectin genotypes and genotype-phenotype relationships in a large cohort of Polish neonates, Hum. Immunol., 70(1):68-72 (2009)
8) Madsen, H. O. et al., Association of mannose-binding lectin deficiency with severe atherosclerosis, Lancet, 352(9132):959-960 (1998)
9) Hegele, R. et al., Mannose-binding lectin gene variation and cardiovascular disease in Canadian Inuit, Clin. Chem., 45, No. 8, pp. 1283-1285, 1999
10) Madsen, H. O. et al., Interplay between promoter and structural gene variants control basal serum level of mannan-binding protein, J. Immunol., 155(6):3013-3020 (1998).
11) Salimans, M. M. M. et al., Association between familial deficiency of mannose-binding lectin and mutations in the corresponding gene and promoter region, Clin. Diagn. Lab. Immunol., 11(4):806-807 (2004)
12) Terai, I. et al., Human serum mannose binding protein (MBP): development of an enzyme-linked immunosorbent assay (ELISA) and determination of levels in serum from 1085 normal Japanese and in some body fluids, Biochem. Med. Metab. Biol., 50:111-119 (1993)
13) Hansen, T. K. et al., Intensive insulin therapy exerts anti-inflammatory effects in critically ill patients and counteracts the effects of low mannose-binding lectin levels, J. Clin. Endocrinol. Metab., 88:1082-1088 (2003)
14) Hanson, T. K., et al., Mannose-binding lectin and Mortality in Type 2 Diabetes, Arch. Intern. Med., 166(18):20072013 (2006)
15) Kuipers, S. et al., A hemolytic assay for the estimation of functional mannose binding lectin (MBL) levels in human serum, J. Immunol. Methods, 268:149-157 (2002)
16) Petersen, K. A. et al., Phase I safety, tolerability, pharmacokinetic study of recombinant human mannan-binding lectin, J. Clin. Immunol., 26:465-75 (2006)
17) Petersen, S. et al., An assay for the mannan-binding lectin pathway of complement activation, J. Immunol. Methods, 257(1-2):107-116 (2001)
18) Hamad, I. et al., Complement activation by PEGylated single-walled carbon nanotubes is independent of C1q and alternative pathway turnover, Mol. Immunol., 45(14):3797-3803 (2008).
19) Herpers, B. L. et al., Hemolytic assay for the measurement of functional human mannose-binding lectin: a modification to avoid interference from classical pathway activation, J. Immunol. Methods, 343(1):61-63 (2009)
20) Walsh, M. C. et al., Mannose-binding lectin is a regulatory of inflammation that accompanies myocardial ischemia and reperfusion injury, J. Immunol., 175 (1):541-546 (2005)
21) Takahashi, K., Mannose-binding lectin and the balance between immune protection and complication, Expert Rev. Anti. Infect. Ther., 9(12):1179-1190 (2011)
22) Troelsen, L. N. et al., Mortality and predictors of mortality in rheumatoid arthritis—a role for mannose-binding lectin?, J. Rheumatol., 37(3):536-42 (2010)
23) Troelsen, L. N. et al., Double role of mannose binding lectin in relation to carotid intima-media thickness in patients with rheumatoid arthritis, Mol. Immunol., 47(4):713-718 (2010)
24) Ohlenschlaeger, T., Mannose-binding lectin variant alleles and the risk of arterial thrombosis in systemic lupus erythematosus, N. Eng. J. Med., 351(3):260-267 (2004)
25) Font, J. et al., Association of mannose-binding lectin gene polymorphisms with antiphospholipid syndrome, cardiovascular disease and chronic damage in patients with systemic lupus erythematosus, Rheumatology (Oxford), 46(1):76-80 (2007)
26) Chang, W. C. et al., Recombinant chimeric lectins consisting of mannose-binding lectin and L-ficolin are potent inhibitors of influenza A virus compared with mannose-binding lectin, Biochem. Pharmacol., 81 (3):388-395 (2011)
27) Haahr-Pedersen, S. et al., Level of complement activity predicts cardiac dysfunction after acute myocardial infarction treated with primary percutaneous coronary intervention, J. Invasive Cardiol., 21(1):13-19 (2009)
28) Biezeveld, M. H. et al., Association of man-nose-binding lectin genotype with cardiovascular abnormalities in Kawasaki disease, Lancet, 361(9365):1268-70 (2003)
29) Testa, L. et al., Pexelizumab in ischemic heart disease: a systematic review and meta-analysis on 15,196 patients, J. Thorac. Cardiovasc. Surg., 136(4):884-893 (2008)
30) Ehrnthaller, C. et al., New insights of an old defense system: structure, function, and clinical relevance of the complement system, Mol. Med., 17(3-4):317-329 (2011)
31) Garred, P. et al., MBL2, FCN1, FCN2 and FCN3—The genes behind the initiation of the lectin pathway of complement, Mol. Immunol., 46(14):2737-44 (2009)
32) Haskell, W. L. et al., Effects of intensive multiple risk factor reduction on coronary atherosclerosis and clinical cardiac events in men and women with coronary artery disease. The Stanford Coronary Risk Intervention Project (SCRIP), Circulation, 89(3):975-990 (1994)
33) Ornish, D. et al., Intensive lifestyle changes for reversal of coronary heart disease, JAMA, 280(23):2001-2007 (1998)
34) Wister, A. et al., One-year follow-up of a therapeutic lifestyle intervention targeting cardiovascular disease risk, CMAJ, 177(8):859-865 (2007)
35) Hansen, T. K. et al., Mannose binding lectin and mortality in type 2 diabetes, Arch. Intern. Med., 166(18):2007-2013 (2006)
36) Hansen, T. K. et al., Association between mannose-binding lectin and vascular complications in type 1 diabetes, Diabetes, 53(6):1570-1576 (2004)
37) Hovind, P. et al., Mannose-binding lectin as a predictor of microalbuminuria in type 1 diabetes: an inception cohort study, Diabetes, 54(5):1523-1527 (2005)
38) Ichinose, K. et al, Recent advancement of understanding of pathogenesis of Type 1 diabetes and potential relevance to diabetic nephropathy, Am. J. Nephrol., 27(6):554-564 (2007)
39) Bouwman, L. H. et al., Elevated levels of mannose-binding lectin at clinical manifestation of type 1 diabetes in juveniles, Diabetes, 54(10):3002-3006 (2005)

What is claimed is:

1. A method for predicting susceptibility or likelihood of a subject having a clinically-relevant mannose-binding lectin (MBL) deficiency to develop cardiodiabetes, comprising:
a) obtaining a measurement value of MBL mass and, optionally, a measurement value of MBL activity level;
b) calculating an MBL-inclusive index score based one or both MBL measurements, wherein the index score calculation involves a mathematical transformation, and
c) comparing the MBL-inclusive index to reference values from a population;
wherein an elevated MBL-inclusive index score correlates with a range in a higher unit of an ordered distribution of the population and indicates that the subject is less susceptible to or has a less likelihood of developing cardiovascular disease and/or cardiodiabetes, and wherein a low MBL-inclusive index score correlates with a range in a lower unit of an ordered distribution of the population and indicates that the subject is more susceptible to or has an increased likelihood of developing cardiovascular disease and/or cardiodiabetes.

2. The method of claim 1, wherein said mathematical transformation involves a logarithmic transformation, a square-root transformation, a quadratic transformation, or combinations thereof.

3. The method of claim 1, wherein an elevated MBL-inclusive index score is classified into tertiles and a score in an upper tertile indicates that the subject is less susceptible to or has a less likelihood of developing cardiovascular disease and/or cardiodiabetes.

4. The method of claim 1, wherein a low MBL-inclusive index score is classified into tertiles and a score in a lower tertile indicates that the subject is more susceptible to or has an increased likelihood of developing cardiovascular disease and/or cardiodiabetes.

5. The method of claim 1, wherein the MBL mass is measured by enzyme-linked immunosorbent assay (ELISA), electrophoresis, double-enzyme immunoassay, immunofluorometry, and/or hemolytic assay.

6. The method of claim 1, wherein the MBL activity level is measured by one or more techniques selected from the group consisting of hemolysis assay, mannan capture assay, micro-organism lysis assay, an assay measuring ability to promote opsonization of a particle or micro-organism, and an assay measuring the production of complement components C4b and/or C3b.

7. The method of claim 1, wherein a low MBL-inclusive score indicates a clinically-relevant MBL deficiency.

8. The method of claim 7, wherein the clinically-relevant MBL deficiency is associated with development of an inflammation, an infection, gestational diabetes, prevalent diabetes, an autoimmunity, a complication from an autoimmune condition or infection, a blood clotting abnormality, an impaired glucose tolerance, an impaired first-phase insulin secretion response, compromised pancreatic beta cell dysfunction, an early insulin resistance, or any form of atherosclerosis.

9. The method of claim 7, wherein the clinically-relevant MBL deficiency identifies a subject at risk for cardiodiabetes, atherosclerosis, heart attack or stroke.

10. The method of claim 1, wherein the MBL-inclusive index score further includes obtaining a measurement value for at least one other biomarker selected from the group consisting of: 1,5 AG; Adiponectin; Alpha hydroxybutyrate; Amylase; Apo A-1; Apo B/ApoA-1 ratio; Apo B-100; apolipoprotein B-48 (ApoB-48); BMI; CD26; C-peptide; C-peptide/Insulin Ratio; C-peptide/Proinsulin ratio; C-reactive protein; Ferritin; Fibrinogen; Free Fatty Acids; Fructosamine; MBL Mass, MBL Activity, Functional MBL/MASP-2 Ratio; glucagon-like peptide 1 (GLP-1); Glucose; Glycation Gap; HbA1c; HDL cholesterol (HDL-C); HDL particle number (HDL-P); HDL particle size; HDL2 levels; HOMA Insulin Resistance Score; Insulin; Insulin Resistance Score; LDL cholesterol (LDL-C); LDL particle number (LDL-P); LDL particle size; LDL Triglycerides; Leptin; Leptin/Adiponectin Ratio; Leptin/BMI ratio; linoleoyl-glycerophosphocholine (L-GPC); LpPLA(2); Mannose; Myeloperoxidase (MPO); OGTT Index; Oleic Acid; Proinsulin; Remnant-like lipoprotein particles (RLPs); RLP-associated cholesterol (RLP-c); small, dense LDL levels (sdLDL); Total Cholesterol; Triglycerides, MBL coding region or promoter genotype; Apo E genotype; Familial Hypercholesterolemia genotype (FH); biomarkers of autoimmunity including but not limited to anti-GAD autoantibodies, anti-islet auto-antibodies, rheumatoid factor, anti-phospholipid antibodies, and anti-nuclear antibodies.

11. The method of claim 1, wherein the MBL-inclusive index score includes the measurements for both MBL mass and MBL activity level.

12. The method of claim 11, wherein the measurements for MBL mass and MBL activity level are transformed as logₙ(MBL mass/MBL activity level).

13. The method of claim 12, wherein the MBL-inclusive index score further includes the measurements for fructosamine, C-peptide, and 1, 5 AG.

14. The method of claim 13, wherein the MBL-inclusive index score comprises the calculation:
$$LN [ \frac{MBL mass * 1 , 5 {AG}^{1.91}}{MBL activity * {Fructosamine}^{10.67} * C \text{-} {peptide}^{2.29}} ]$$

15. The method of claim 12, wherein the MBL-inclusive index score is calculated by
i. dividing the measurement value of MBL mass with the measurement value of MBL activity level;
ii. mathematically incorporating the measurement of at least one other biomarker; and
iii. logarithmically transforming the outcome generated from the dividing and mathematically incorporating steps.

16. The method of claim 1, wherein the method further comprises screening for a genotype in an MBL coding sequence and its promoter region.

17. The method of claim 1, wherein the method further comprises measuring an amount of an MBL-binding serine protease, genotyping an MASP coding region, genotyping an MASP promoter region, or combinations thereof.

18. The method of claim 1, wherein the susceptibility or likelihood of the subject to have cardiovascular disease and/or cardiodiabetes is low, medium or high.

19. The method of claim 1, wherein a high MBL-inclusive index score indicates a higher risk of having or developing cardiovascular disease in a subject that has an autoimmune disease or condition.

20. The method of claim 1, further comprising administering a therapeutic regimen for the treatment or prevention of cardiovascular disease or cardiodiabetes.

21. The method of claim 20, wherein the therapeutic regimen is selected from the group consisting of (i) administration of a recombinant human MBL, plasma-derived MBL or an MBL analogue and/or inhibitor; (ii) administration of lipid-modulating compounds for aggressive management of LDL and Apo-B; (iii) diet and lifestyle intervention; (iv) administration of antibiotics and/or anti-viral agents; (v) administration of immuno-modulating therapies; (vi) administration of coagulation therapies; (vii) administration of therapeutics that modify the complement cascade; (viii) an antihypertensive therapy; (ix) an antibdiabetic therapy; (x) other drug-based and lifestyle-based therapeutic interventions; and a combination thereof.

22. The method of claim 20, wherein the therapeutic regimen further includes administration of drugs or supplements; treatment for chronic infections; referral to a healthcare specialist or related specialist based on the determination of the risk levels; recommendations on making or maintaining lifestyle choices; or combinations thereof.

23. The method of claim 22, wherein the drugs or supplements are selected from the group consisting of (i) administration of a recombinant human MBL, plasma-derived MBL or an MBL analogue and/or inhibitor; (ii) administration of lipid-modulating compounds for aggressive management of LDL and Apo-B; (iii) diet and lifestyle intervention; (iv) administration of antibiotics and/or anti-viral agents; (v) administration of immuno-modulating therapies; (vi) administration of coagulation therapies; (vii) administration of therapeutics that modify the complement cascade; (viii) an antihypertensive therapy; (ix) an antibdiabetic therapy; (x) other drug-based and lifestyle-based therapeutic interventions; and a combination thereof.

24. A method for predicting susceptibility or likelihood of a subject having a clinically-relevant mannose-binding lectin (MBL) deficiency to develop cardiodiabetes, comprising:
a. obtaining measurement values of MBL mass and MBL activity level;
b. obtaining measurement values for Fructosamine, C-peptide, and 1, 5 AG;
c. calculating an MBL-inclusive index score based the measurements obtained in steps (a) and (b) using the following equation:
$$LN [ \frac{MBL mass * 1 , 5 {AG}^{1.91}}{MBL activity * {Fructosamine}^{10.67} * C \text{-} {peptide}^{2.29}} ] ;$$
d. comparing the MBL-inclusive index to reference values from a population;
wherein an elevated MBL-inclusive index score correlates with a range in a higher unit of an ordered distribution of the population and indicates that the subject is less susceptible to or has a less likelihood of developing cardiovascular disease and/or cardiodiabetes, and wherein a low MBL-inclusive index score correlates with a range in a lower unit of an ordered distribution of the population and indicates that the subject is more susceptible to or has an increased likelihood of developing cardiovascular disease and/or cardiodiabetes.

25. A method for predicting susceptibility or likelihood of a subject having a clinically-relevant mannose-binding lectin (MBL) deficiency to develop cardiodiabetes, comprising:
a. obtaining measurement values of MBL mass and MBL activity level;
b. calculating an MBL-inclusive index score based the measurements obtained in step (a) using the following equation:
$$i . log [ \frac{MBL mass}{MBL activity} ] ;$$
c. comparing the MBL-inclusive index to reference values from a population;
wherein an elevated MBL-inclusive index score correlates with a range in a higher unit of an ordered distribution of the population and indicates that the subject is less susceptible to or has a less likelihood of developing cardiovascular disease and/or cardiodiabetes, and wherein a low MBL-inclusive index score correlates with a range in a lower unit of an ordered distribution of the population and indicates that the subject is more susceptible to or has an increased likelihood of developing cardiovascular disease and/or cardiodiabetes.
